(19) United States
(12) Patent Application Publication
(10) Pub. No.: US 2006/0247778 A1
(43) Pub. Date: Nov. 2, 2006

(54) Intradiscal devices including spacers facilitating posterior-lateral and other insertion approaches

(76) Inventors: Bret A. Ferree, Cincinnati, OH (US); David Tompkins, Milford, OH (US)

Correspondence Address: John G. Posa;Gifford, Krass, Groh, Sprinkle,, Anderson & Citkowski, P.C., PO Box 7021, Troy, MI, 48007, US

(21) Appl. No.: 11/391,966

(22) Filed: Mar. 29, 2006

Related U.S. Application Data
(60) Provisional application No. 60/666,069, filed on Mar. 29, 2005.

(30) Foreign Application Priority Data
Jan. 26, 2005 (WO) PCT/DE05/00105

(51) Int. Cl. A61F 2/44; A61F 2/28

(52) U.S. Cl. 623/17.14; 623/23.61; 623/17.16

(57) ABSTRACT

Apparatus and methods are used to expand and/or connect disc replacement devices in situ, allowing such devices to be inserted through smaller openings including posterior as well as an anterior approaches to the spine. Other embodiments reside in nucleus replacements that do not expand within the disc space, providing improved longevity compared to existing NRs. Embodiments of the invention may be used in the cervical, thoracic, or lumbar spine. The invention may also be used in other joints such as, the knee, prosthetic knees, prosthetic hips, or other joints in the body.

### REFERENCE TO RELATED APPLICATION

This application claims priority to U.S. Provisional Patent Application Ser. No. 60/666,069, filed Mar. 29, 2005, the entire content of which is incorporated herein by reference.

### FIELD OF THE INVENTION

This invention relates generally to intradiscal devices and. in particular, to artificial disc replacements (ADRs) and nucleus replacements (NRs) that do not expand within the disc space, providing improved insertion strategies and/or longevity.

### BACKGROUND OF THE INVENTION

The human intervertebral disc is an oval to kidney bean shaped structure of variable size depending on the location in the spine. The outer portion of the disc is known as the annulus fibrosis (AF). The AF is formed of 10 to 60 fibrous bands. The fibers in the bands alternate their direction of orientation by 30 degrees between each band. The orientation serves to control vertebral motion (one half of the bands tighten to check motion when the vertebra above or below the disc are turned in either direction).

The AF contains the nucleus. The nucleus pulpous serves to transmit and dampen axial loads. A high water content (70-80 percent) assists the nucleus in this function. The water content has a diurnal variation. The nucleus imbibes water while a person lies recumbent. Activity squeezes fluid from the disc. Nuclear material removed from the body and placed into water will imbibe water swelling to several times its normal size. The nucleus comprises roughly 50 percent of the entire disc. The nucleus contains cells (chondrocytes and fibrocytes) and proteoglycans (chondroitin sulfate and keratin sulfate). The cell density in the nucleus is on the order of 4,000 cells per micro liter.

The disc changes with aging. As a person ages the water content of the disc falls from approximately 85 percent at birth to 70 percent in the elderly. The ratio of chondroitin sulfate to keratin sulfate decreases with age. The ratio of chondroitin 6 sulfate to chondroitin 4 sulfate increases with age. The distinction between the annulus and the nucleus decreases with age. These changes are known as disc degeneration. Generally disc degeneration is painless.

Premature or accelerated disc degeneration is known as degenerative disc disease. A large portion of patients suffering from chronic low back pain are thought to have this condition. As the disc degenerates, the nucleus and annulus functions are compromised. The nucleus becomes thinner and less able to handle compression loads. The annulus fibers become redundant as the nucleus shrinks. The redundant annular fibers are less effective in controlling vertebral motion. The disc pathology can result in: 1) bulging of the annulus into the spinal cord or nerves; 2) narrowing of the space between the vertebra where the nerves exit; 3) tears of the annulus as abnormal loads are transmitted to the annulus and the annulus is subjected to excessive motion between vertebra; and 4) disc herniation or extrusion of the nucleus through complete annular tears.

Current surgical treatments of disc degeneration are destructive. One group of procedures removes the nucleus or a portion of the nucleus; lumbar discectomy falls in this category. A second group of procedures destroy nuclear material; Chymopapin (an enzyme) injection, laser discectomy, and thermal therapy (heat treatment to denature proteins) fall in this category. A third group, spinal fusion procedures either remove the disc or the disc's function by connecting two or more vertebra together with bone. These destructive procedures lead to acceleration of disc degeneration. The first two groups of procedures compromise the treated disc. Fusion procedures transmit additional stress to the adjacent discs. The additional stress results in premature disc degeneration of the adjacent discs.

Prosthetic disc replacement offers many advantages. The prosthetic disc attempts to eliminate a patient's pain while preserving the disc's function. Current prosthetic disc implants, however, either replace the nucleus or the nucleus and the annulus. Both types of current procedures remove the degenerated disc component to allow room for the prosthetic component.

Artificial Disc Replacements (ADRs) known as Nucleus Replacements (NRs) are often inserted from a posterior approach to the spine. Nucleus replacements are generally designed to enlarge within the disc space. The small initial size of NRs facilitates insertion of NRs from a posterior approach. Nucleus replacements are soft, cushion-like devices that fit between the vertebral endplates (VEPs.). Nucleus Replacements are not attached to the VEPs. The small initial size of NRs and the flexibility of NRs minimize nerve injury during insertion of the devices from a posterior approach to the spine. Only a limited number of biocompatible materials expand within the disc space. Materials that expand within the disc space are less robust than materials that do not swell or expand in the disc space. Consequently, current NRs will likely to wear out during a patient's lifetime.

Prior art ADRs known as Total Disc Replacements (TDRs) have rigid endplates that are attached to the vertebra above and below the TDR. The rigid TDRs do not expand within the disc space. The large size of TDRs and the rigidity of TDRs make insertion from a posterior approach to the spine dangerous. Metal TDRs will likely last a patient's lifetime. Nucleus replacements dampen loads that are applied to the spine. Total disc replacements do not dampen loads that are applied to the spine.

### SUMMARY OF THE INVENTION

The present invention improves upon prior art artificial disc replacements (ADRs) in several important ways. First, the invention may be used to expand TDRs within the disc space, allowing such devices to be inserted from a posterior as well as an anterior approach to the spine. Expanding TDRs may be inserted through smaller openings in the Annulus Fibrosus (AF).

The invention may further be used to design Nucleus Replacements (NRs) that do not expand within the disc space, providing improved longevity compared to existing NRs.

Embodiments of the invention may be used in the cervical, thoracic, or lumbar spine. The invention may also be used in other joints such as, the knee, prosthetic knees, prosthetic hips, or other joints in the body. The non-expanding NRs are preferably inserted using the annulus preserving methods taught in my co-pending application U.S. patent application Ser. No. 10/421,434, the entire content of which is incorporated herein by reference.

Nucleus replacement embodiments of the device are preferably made form polymers including, but not limited to, BioSpan, Bionate, Elasthane, PurSil, CarboSIl, Calo-Mer from the Polymer Technology Group in Berkley Calif.; other polyurethanes including solution polyurethanes, thermoplastic polyurethanes, foam polyurethanes; silicones, thermoplastic silicone urethane copolymers; shape memory thermoplastics; hydrocarbon based polymers; C-Flex, hydrogels, Estane (Goodrich), Texin (Bayer), Roylar (Uniroyal), Chromoflex (Cardiotech), and Biomer (Thoratec). Total disc embodiments of the device are preferably made of biocompatible materials such as titanium, chrome cobalt, and ceramic.

### BRIEF DESCRIPTION OF THE DRAWINGS

FIG. 1 is an axial cross section of a lumbar disc and the soft tissues surrounding the spine;

FIG. 2A is a coronal cross section of an embodiment of the present invention and the spine;

FIG. 2B is a lateral view of the spine and the embodiment of the present invention drawn in FIG. 2A;

FIG. 3A is a view of the top of the embodiment of the present invention drawn in FIG. 2A;

FIG. 3B is a view of the top of a component that fits into the embodiment of the present invention drawn in FIG. 3A;

FIG. 3C is a view of the top of the embodiments of the present invention drawn in FIGS. 3A and 3B;

FIG. 3D is a view of the top of the assembled device of the embodiment of the present invention drawn in FIG. 3C;

FIG. 4A is an axial cross section of a disc and a view of the top of an alternative embodiment of the present invention drawn in FIG. 3D;

FIG. 4B is an axial cross section of a disc and a view of the top of the embodiment of the present invention drawn in FIG. 4A;

FIG. 4C is an axial view of the disc and the assembled device drawn inn FIG. 4B;

FIG. 4D is a lateral view of the embodiment of the present invention drawn in FIG. 4C;

FIG. 5A is a view of the top of an alternative embodiment of the present invention drawn in FIG. 4C;

FIG. 5B is a top view of an exploded view of the embodiment of the present invention drawn in FIG. 5A;

FIG. 5C is a lateral view of the assembled device drawn in FIG. 5A;

FIG. 6A is a top view of an alternative embodiment of the present invention drawn in FIG. 5A;

FIG. 6B is an exploded view of the embodiment of the present invention drawn in FIG. 6A;

FIG. 6C is a view of the inferior surface of the unassembled device drawn in FIG. 6A;

FIG. 6D is a view of the inferior surface of an assembled device drawn in FIG. 6A;

FIG. 6E is a lateral view of the embodiment of the present invention drawn in FIG. 6A;

FIG. 7A is a top view of an alternative embodiment of the present invention;

FIG. 7B is a view of the top of a wedge component;

FIG. 7C is a view of the top of the top of an embodiment of the device assembled by inserting the component drawn in FIG. 7B into the component drawn in FIG. 7A;

FIG. 7D is a lateral view of the embodiment of the present invention drawn in FIG. 7C;

FIG. 8 is the view of the top of an alternative embodiment of the present invention and an axial cross section of a disc;

FIG. 9 is a view of the top of an alternative embodiment of the present invention drawn in FIG. 8 and an axial cross section of a disc;

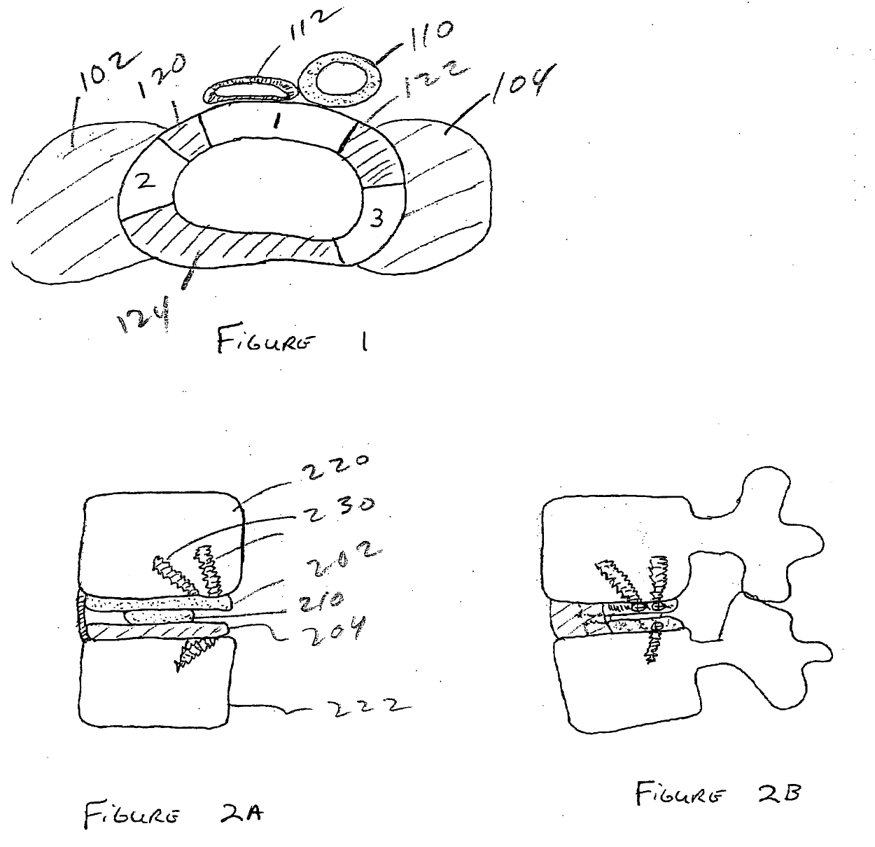

FIG. 1OA is an exploded view of the top of an alternative embodiment of the present invention including anterior and posterior components that slide along one

FIG. 10A is an exploded view of the top of an alternative embodiment of the

FIG. 1OB is a view of the top of the embodiment of the present invention drawn in FIG. 10A;

FIG. 10B is a view of the top of the embodiment of the present invention drawn in FIG. 10A and an axial cross section of a disc;

FIG. 10D is a lateral view of the embodiment of the present invention drawn in Figure 10B;

FIG. 11A is an exploded view of the top of an alternative embodiment of the present invention;

FIG. 11B is a view of the top of the embodiment of the present invention drawn in FIG. 11A;

FIG. 12A is a view of the top of an alternative embodiment of the present invention drawn in FIG. 11B;

FIG. 12B is a view of the top of the embodiment of the present invention drawn in FIG. 12A;

FIG. 13A is a view of the top of an alternative embodiment of the present invention drawn in FIG. 12A and an axial cross section of a disc;

FIG. 13B is an exploded view of the top of the embodiment of the present invention drawn in FIG. 13A;

FIG. 13C is a view of the bottom of the embodiment of the present invention drawn in FIG. 13B;

FIG. 13D is a view of the bottom of the embodiment of the present invention drawn in FIG. 13A;

FIG. 13E is an exploded view of the bottom of an alternative embodiment of the present invention drawn in FIG. 13C;

FIG. 13F is view of the bottom of the embodiment of the present invention drawn in FIG. 13E;

FIG. 14A is a view of the top of an alternative embodiment of the present invention;

FIG. 14B is a view of the top of the embodiment of the present invention drawn in FIG. 14A;

FIG. 14C is a view of the top of the embodiment of the present invention drawn in FIG. 14A and an axial cross section of the disc;

FIG. 15A is a view of the top of an alternative embodiment of the present invention drawn in FIG. 14A;

FIG. 15B is a view of the top of an alternative embodiment of the present invention drawn in FIG. 15A;

FIG. 16A is an exploded view of the top of an alternative embodiment of the present invention;

FIG. 16B is an exploded view of the top of the embodiment of the present invention drawn in FIG. 16A and an axial cross section of a disc;

FIG. 16C is a view of the top of the embodiment of the present invention drawn in FIG. 16B and an axial cross section of a disc;

FIG. 16D is a view of the top of an alternative embodiment of the anterior TDR component drawn in FIG. 16A;

FIG. 17A is an exploded view of the top of an alternative embodiment of the present invention drawn in FIG. 16A;

FIG. 17B is a view of the top of the embodiment of the present invention drawn in FIG. 17A;

FIG. 18A is an exploded view of an alternative embodiment of the present invention;

FIG. 18B is a view of the top of the embodiment of the present invention drawn in FIG. 18A;

FIG. 19A is an exploded view of an alternative embodiment of the present invention and an axial cross section of the disc;

FIG. 19B is a view of the top of the embodiment of the present invention drawn in FIG. 19A and an axial cross section of the disc;

FIG. 20A is lateral view of an alternative embodiment of the present invention drawn in FIG. 13A;

FIG. 20B is a lateral view of the embodiment of the present invention drawn in FIG. 20A with the TDR drawn in its contracted position;

FIG. 20C is a view of the top of the embodiment of the present invention drawn in FIG. 20A with the TDR drawn in its extended position;

FIG. 20D is a view of the top of the embodiment of the present invention drawn in FIG. 20B with the TDR drawn in its contracted position;

FIG. 20E is a view of the bottom of the embodiment of the present invention drawn in FIG. 20C;

FIG. 20F is an exploded view of the bottom of the embodiment of the present invention drawn in FIG. 20D;

FIG. 20G is a view of the bottom of an alternative embodiment of the present invention drawn in FIG. 20G;

FIG. 21A is an exploded view of bottom of an alternative embodiment of the present invention;

FIG. 21B is a view of the bottom of an alternative embodiment of the present invention drawn in FIG. 21A;

FIG. 22 is a view of the bottom of an alternative embodiment of the present invention drawn in FIG. 21B;

FIG. 23A is an anterior view of the embodiment of the present invention wherein the retractable members extend from the top to the bottom of each TDR EP;

FIG. 23B is an anterior view of an alternative embodiment of the present invention;

FIG. 24A is an exploded view of top of an alternative embodiment of the invention drawn in FIG. 20A;

FIG. 24B is a view of the top of the embodiment of the invention drawn in FIG. 24A;

FIG. 24C is view of the bottom of an alternative embodiment of the invention wherein the retractable and articulating components have holes;

FIG. 25A is a view of the top of an alternative embodiment of the invention;

FIG. 25B is a view of the top of the wedge component drawn in FIG. 25A;

FIG. 25C is a lateral view of the wedge component drawn in FIG. 25B;

FIG. 26A is an axial cross section of a disc, a psoas muscle, and a novel articulating retractor;

FIG. 26B is an axial cross section of a disc, a psoas muscle, and the embodiment of the invention drawn in FIG. 26A;

FIG. 27A is an oblique view of an alternative embodiment of the invention;

FIG. 27B is a coronal cross section the embodiment of the invention drawn in FIG. 27A;

FIG. 27C is an axial cross section of the disc and a view of the top of the embodiment drawn in FIG. 27A;

FIG. 27D is a coronal cross section of the spine and an anterior view of the embodiment of the invention drawn in FIG. 27C;

FIG. 28A is an oblique view of an alternative embodiment of the invention;

FIG. 28B is a coronal cross section of the embodiment of the invention drawn in FIG. 28A;

FIG. 28C is an axial cross section of a disc and a view of the top of the embodiment of the invention drawn in FIG. 28A;

FIG. 28D is a coronal cross section of the embodiment of the invention drawn in FIG. 28B;

FIG. 29A is a lateral view of an alternative embodiment of the invention with cylinder shaped projections on the top and the bottom of the device;

FIG. 29B is a view of the top of the embodiment of the invention drawn in FIG. 29A;

FIG. 30A is a lateral view of an alternative embodiment of the invention drawn in FIG. 29A;

FIG. 30B is a view of the top of the embodiment of the invention drawn in FIG. 30A;

FIG. 31A is lateral view of an alternative embodiment of the invention in the form of a device with holes that extend into the sides of the NR;

FIG. 31B is a view of the top of the embodiment of the invention drawn in FIG. 31A;

FIG. 31C is an axial cross section of the embodiment of the invention drawn in FIG. 31B;

FIG. 31D is a coronal cross section of the embodiment of the device drawn in FIG. 31C;

FIG. 32 is an axial cross section through an alternative embodiment of the invention;

FIG. 33 is an axial cross section through an alternative embodiment of the invention drawn in FIG. 32;

FIG. 34 is an axial cross section of an alternative embodiment of the invention wherein the holes in the NR course form left to right and from anterior to posterior;

FIG. 35 is a lateral view of the embodiment of the invention drawn in FIG. 34;

FIG. 36 is a lateral view of an alternative embodiment of the invention wherein the holes are circular in cross section;

FIG. 37 is a view of the top of an alternative embodiment of the invention including a large projection from the top and bottom of the posterior portion of the NR;

FIG. 38 is a view of the top of an alternative embodiment of the invention wherein large four-pointed, star-like components project from the top and/or the bottom of the NR;

FIG. 39 is lateral view of an alternative embodiment of the invention wherein an elastic band surrounds the periphery of the NR;

FIG. 40 is a sagittal cross section of the spine, a NR or TDR, and alternative embodiment of the invention;

FIG. 41A is an anterior view of an alternative embodiment of the invention;

FIG. 41B is an axial cross section through the embodiment of the invention drawn in FIG. 41A;

FIG. 41C is an anterior view of an alternative embodiment of the invention drawn in FIG. 41A;

FIG. 42A is a lateral view of an alternative embodiment of the invention drawn in FIG. 41A;

FIG. 42B is a view of the top the embodiment of the invention drawn in FIG. 42A;

FIG. 43A is a lateral view of an alternative embodiment of the invention wherein the NR has holes or slots on the top and the bottom of the device;

FIG. 43B is a view of the top of the embodiment of the invention drawn in FIG. 43A;

FIG. 44A is a view of the front of an alternative embodiment of the invention wherein, like the NR in FIG. 41A;

FIG. 44B is an axial cross section of the embodiment of the invention drawn in FIG. 44A;

FIG. 44C is a sagittal cross section of the embodiment of the invention drawn in FIG. 44B;

FIG. 45A is a lateral view of an alternative embodiment of the invention wherein the softer material passes through a tube-shaped opening in the posterior portion of the device;

FIG. 45B is a view of the front of the embodiment of the invention drawn in FIG. 45A;

FIG. 45C is a view of the back of the embodiment of the invention drawn in FIG. 45A;

FIG. 45D is a view of the top of the embodiment of the invention drawn in FIG. 45A;

FIG. 46A is a view of the top of an alternative embodiment of the invention drawn in FIG. 19A;

FIG. 46B is an anterior view of the embodiment of the invention drawn in FIG. 46A;

FIG. 46C is an exploded view of the top of the embodiment of the invention drawn in FIG. 46A;

FIG. 46D is an axial cross section of a disc and an exploded view of the top of the embodiment of the invention drawn in FIG. 46C;

FIG. 47A is an axial cross section of an alternative embodiment of the invention drawn in FIG. 41B; and

FIG. 47B is a view of the anterior-lateral portion of the invention drawn in FIG. 47A.

### DETAILED DESCRIPTION OF THE INVENTION

FIG. 1 is an axial cross section of a lumbar disc and the soft tissues surrounding the spine. The large crescent shaped structures 102, 104 on either side of the disc represent the psoas muscle. The aorta is depicted at 110, and the vena cava at 112. The portions of the disc at 120, 122, 124 represent the annulus fibrosis (AF).

The area labeled as “1” is the portion of AF removed for insertion of an ADR through an anterior approach to the spine. The area of the drawing labeled as “2” is the portion of AF removed for insertion of an ADR through a lateral approach to the spine. The area of the drawing labeled as “3” is the portion of AF removed for insertion of an ADR through a posterior-lateral approach to the spine. The preferred embodiments of the invention are inserted through posterior-lateral approach to the spine, though the other approaches may also be used. For example, the anterior approach may be the preferred approach for insertion of cervical embodiments of the invention.

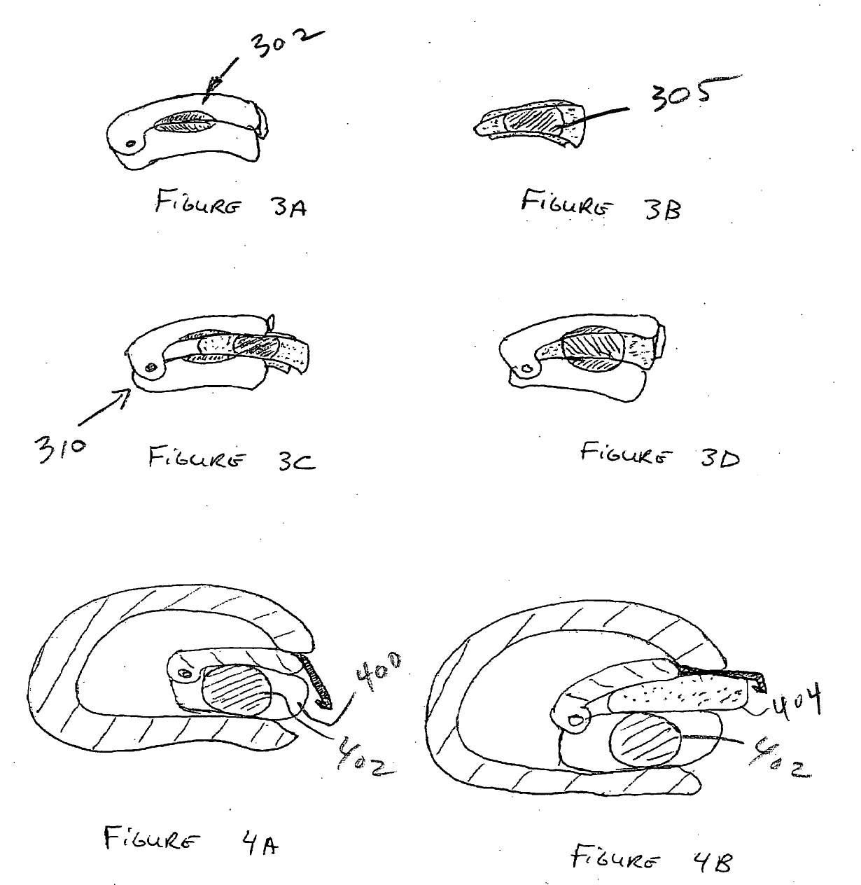

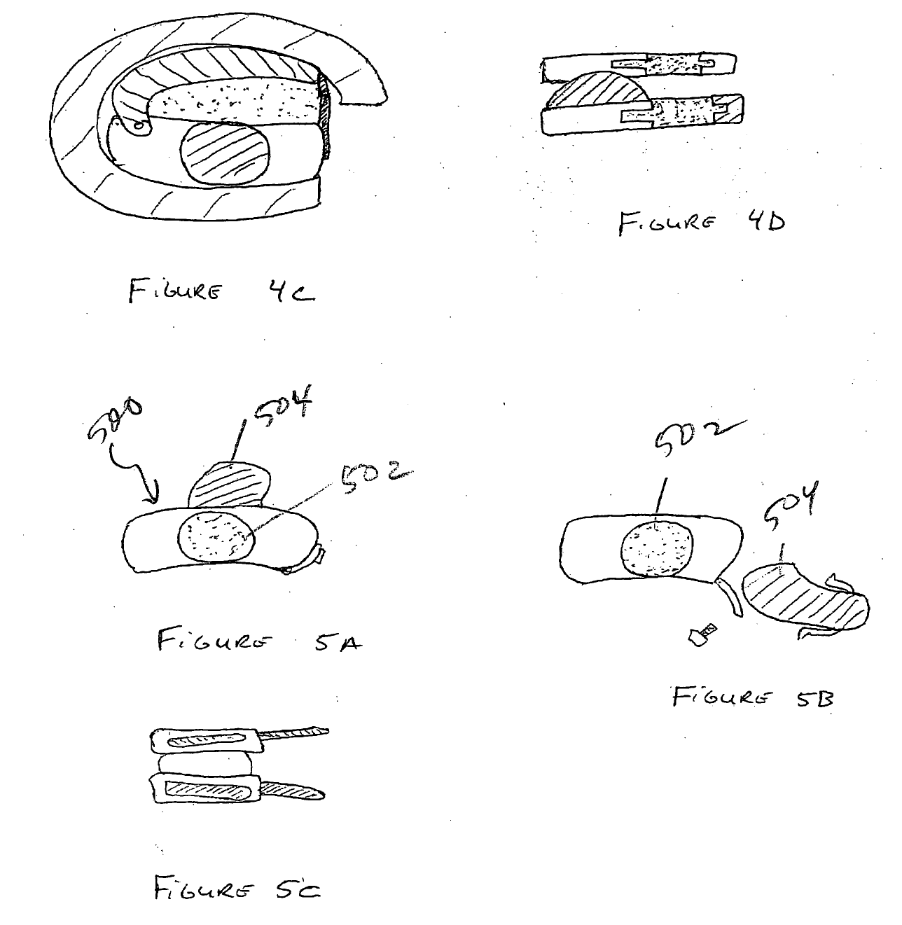

FIG. 2A is a coronal cross section of an embodiment of the invention and the spine. The total disc replacement (TDR) has upper and lower endplates 202, 204 that articulate relative to one another. For example, the endplates may articulate through a spherical joint 210 between the components. The endplates are attached to the vertebrae 220, 222. For example, screws 230 may pass through the TDR endplates into the vertebrae. FIG. 2B is a lateral view of the spine and the embodiment of the invention drawn in FIG. 2A. Note that the TDR endplates extend anteriorly relative to the window cut in the AF.

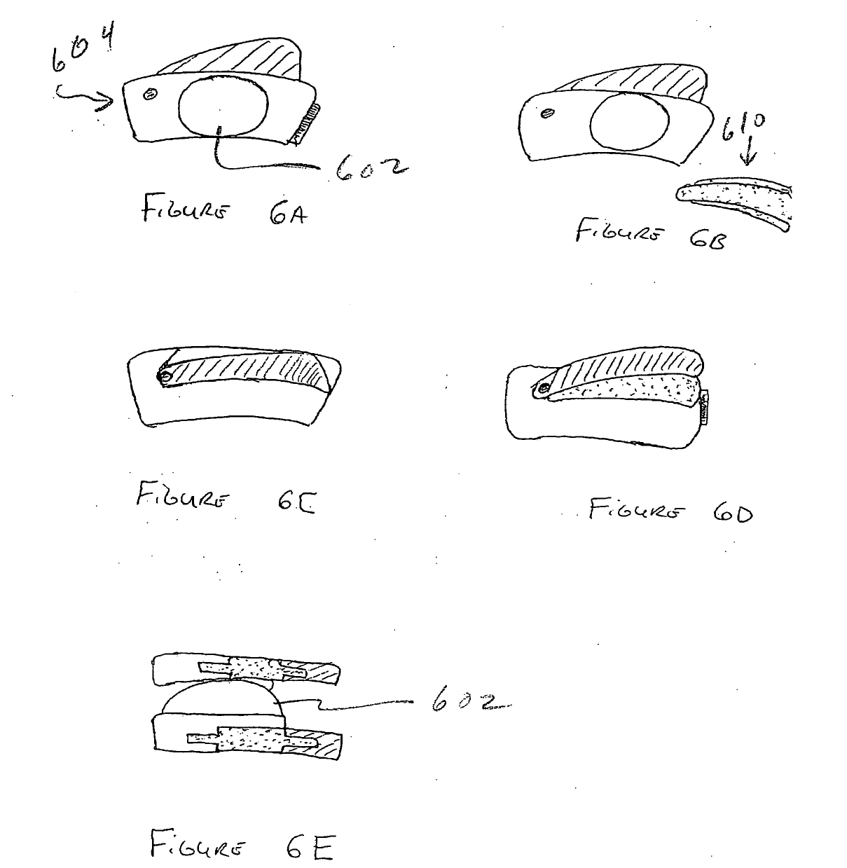

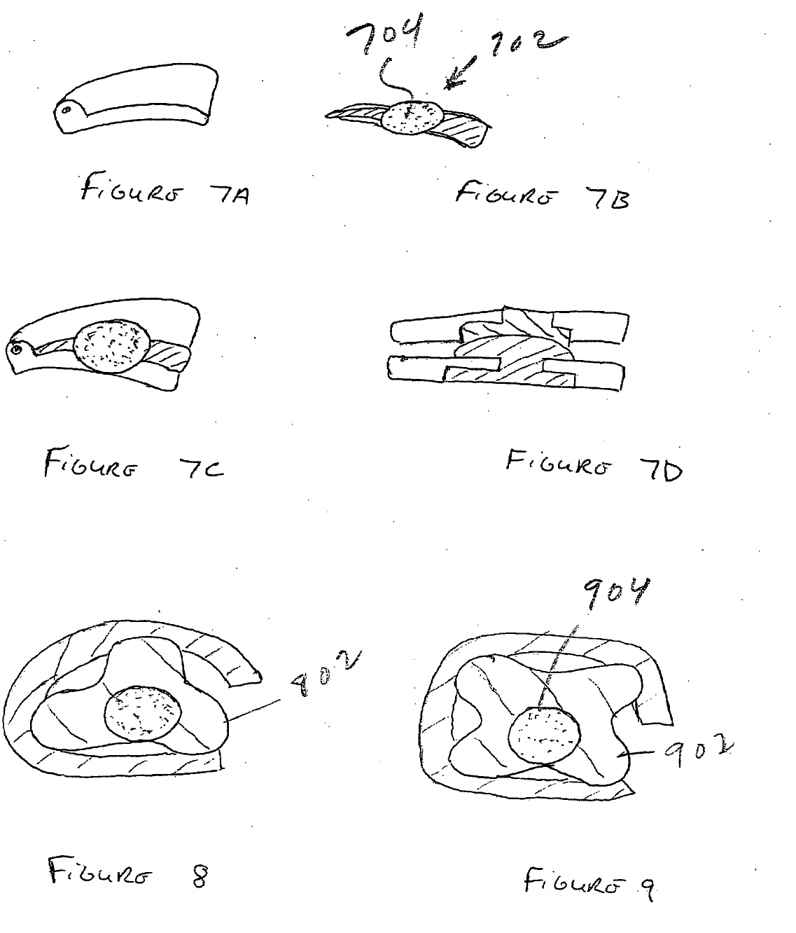

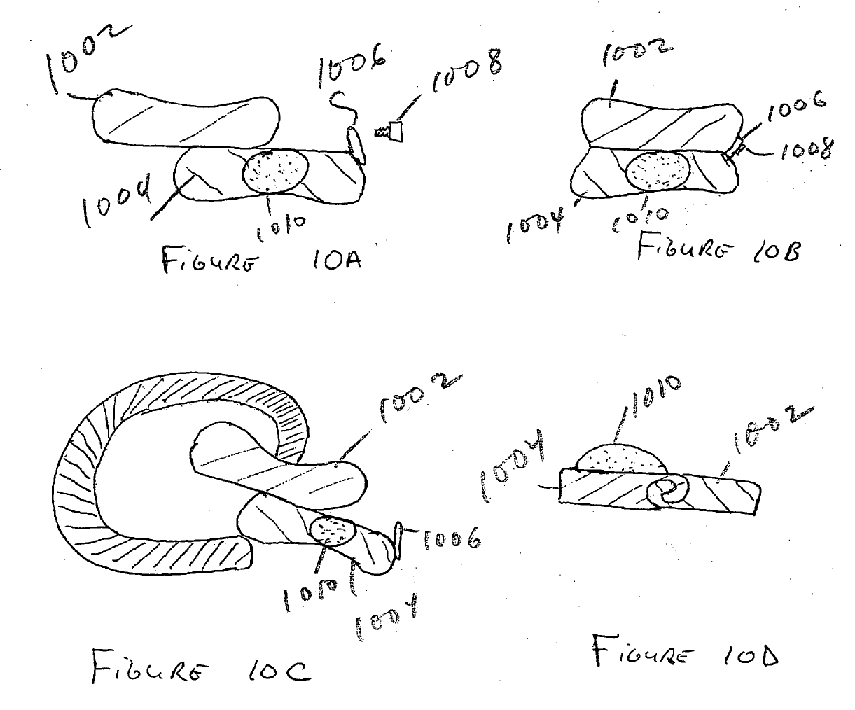

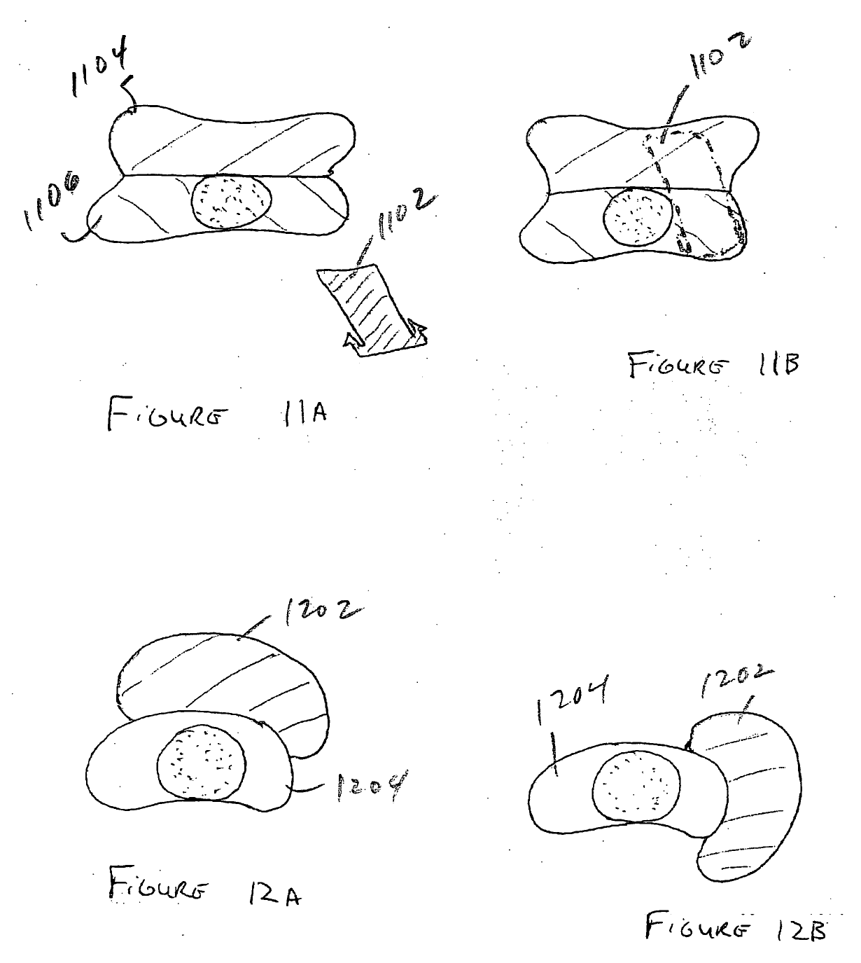

FIG. 3A is a view of the top of the embodiment of the invention drawn in FIG. 2A. The device is drawn in its first, collapsed, shape. The ellipse 302 represents portions of a spherical joint. The portion could be the concavity or the convexity of the spherical joint. FIG. 3B is a view of the top of a component that fits into the embodiment of the invention drawn in FIG. 3A. The center area 305 of the device has a portion of the spherical joint.

FIG. 3C is a view of the top of the embodiments of the invention drawn in FIGS. 3A and 3B. The component drawn in FIG. 3B slides into the component drawn in FIG. 3A, forcing apart the anterior and posterior halves. The anterior and posterior halves of the component drawn in FIG. 3A may be connected by a hinge joint 310.

FIG. 3D is a view of the top of the assembled device of the embodiment of the invention drawn in FIG. 3C. The components assemble to form an articulating surface, preferably spherical. The assembled device is wider from anterior to posterior than unassembled components drawn in FIGS. 3A and 3B. A latch or other fastening mechanism may be used to hold the assembled device together.

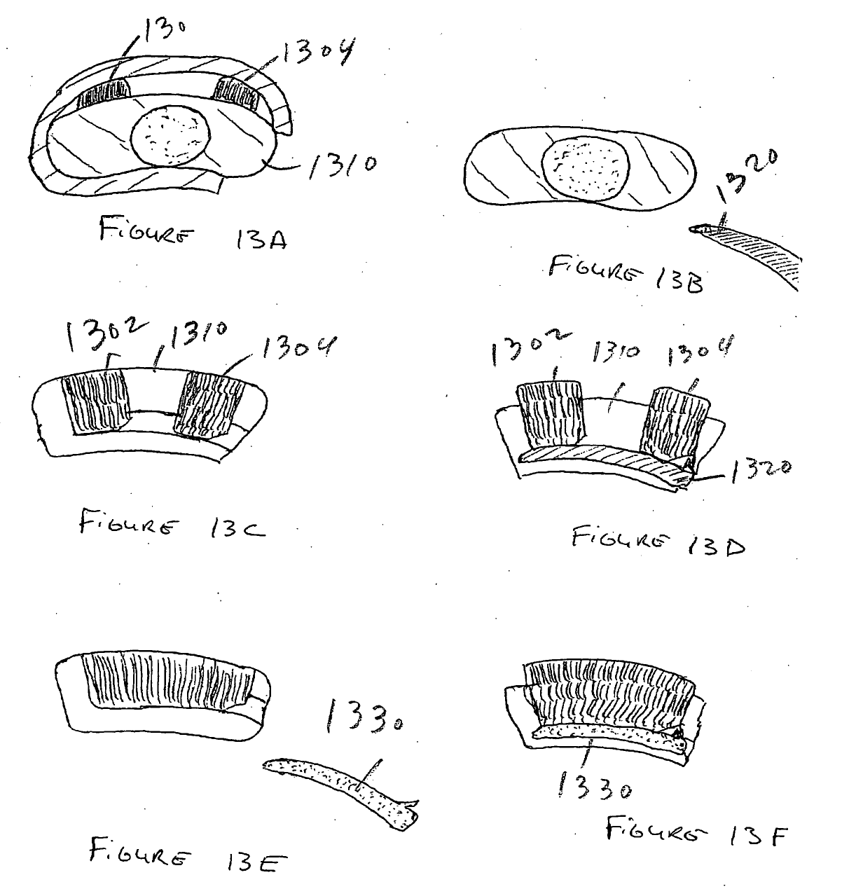

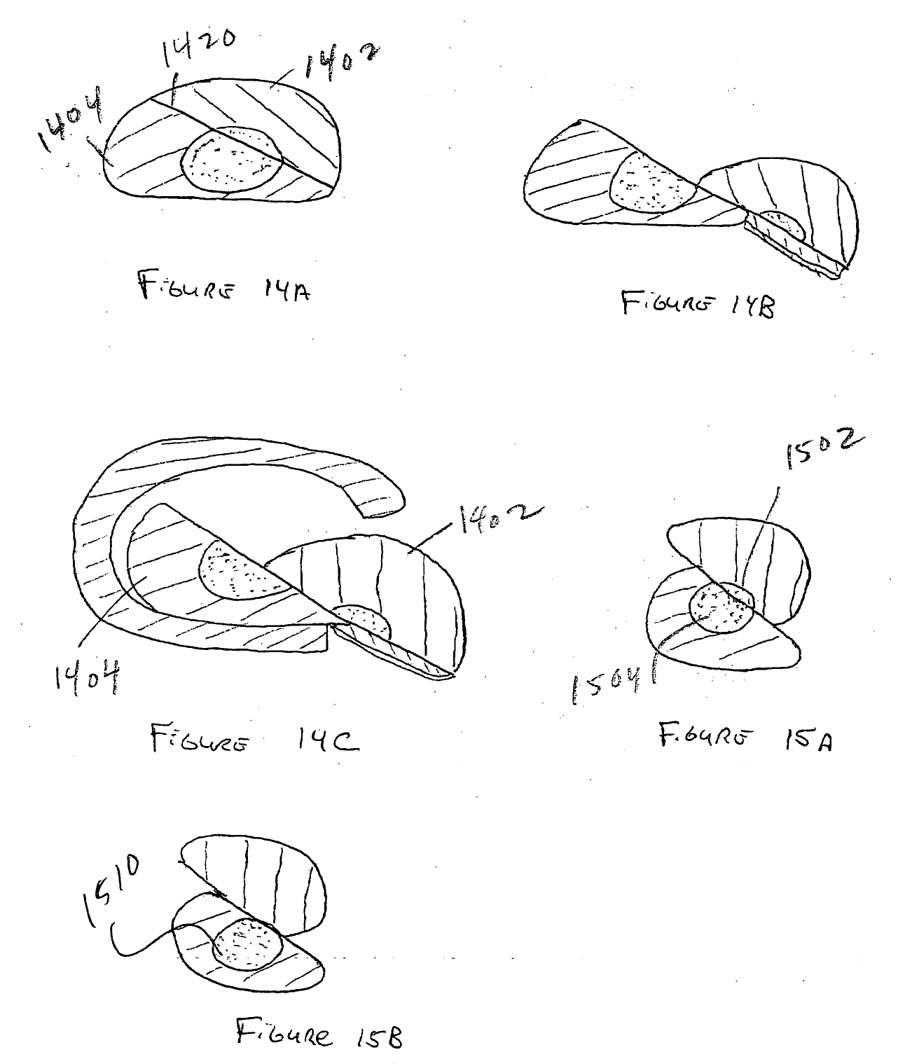

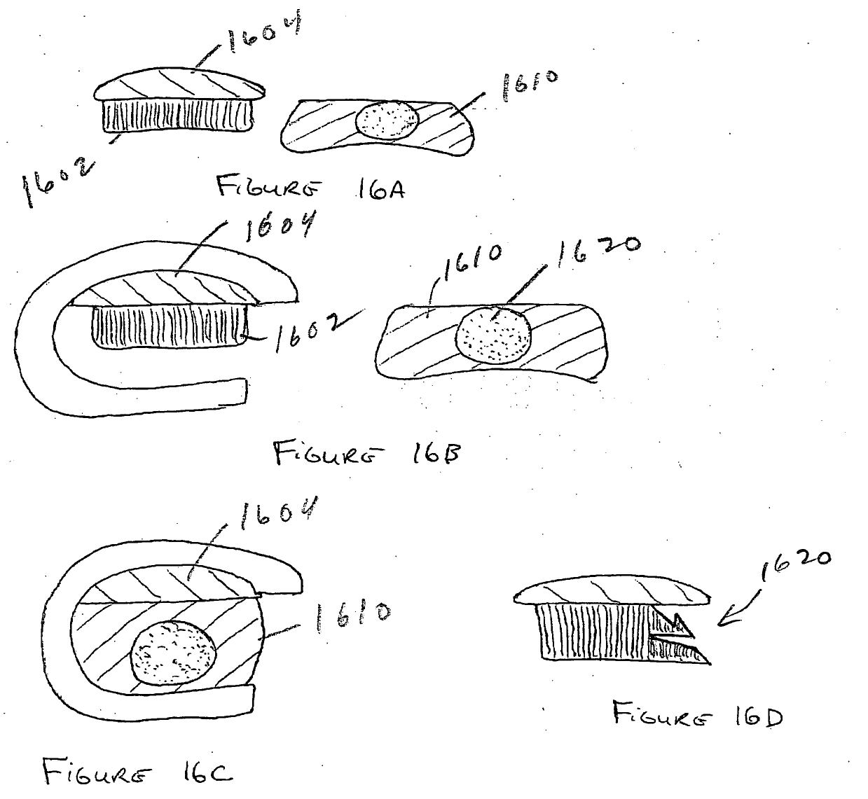

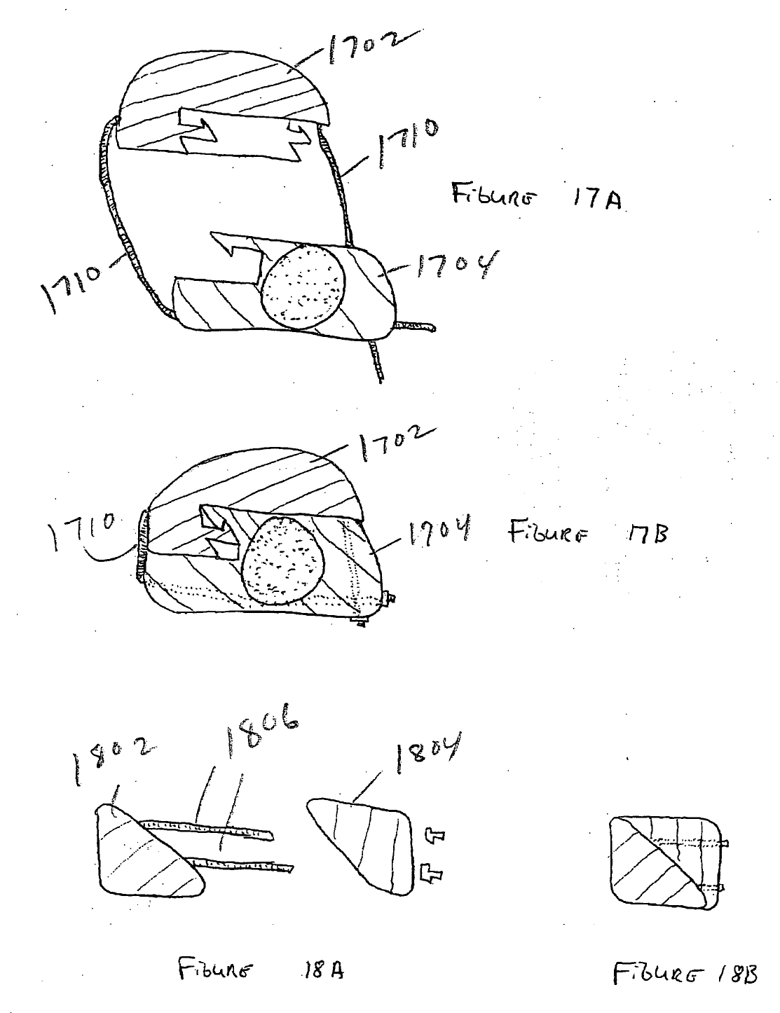

FIG. 4A is an axial cross section of a disc and a view of the top of an alternative embodiment of the invention. The posterior half 400 of the device has a spherical articulating surface 402. The device is inserted through an opening in the posterior-lateral portion of the AF.

FIG. 4B is an axial cross section of a disc and a view of the top of the embodiment of the invention drawn in FIG. 4A. A second component 404 is placed into the first component after the first component is placed into the disc space. The second component forces the device to enlarge in the anterior to posterior direction. FIG. 4C is an axial view of the disc and the assembled device drawn in FIG. 4B. The device fits within the AF and is co-extensive with most of the vertebral endplates. FIG. 4D is a lateral view of the embodiment of the invention drawn in FIG. 4C.

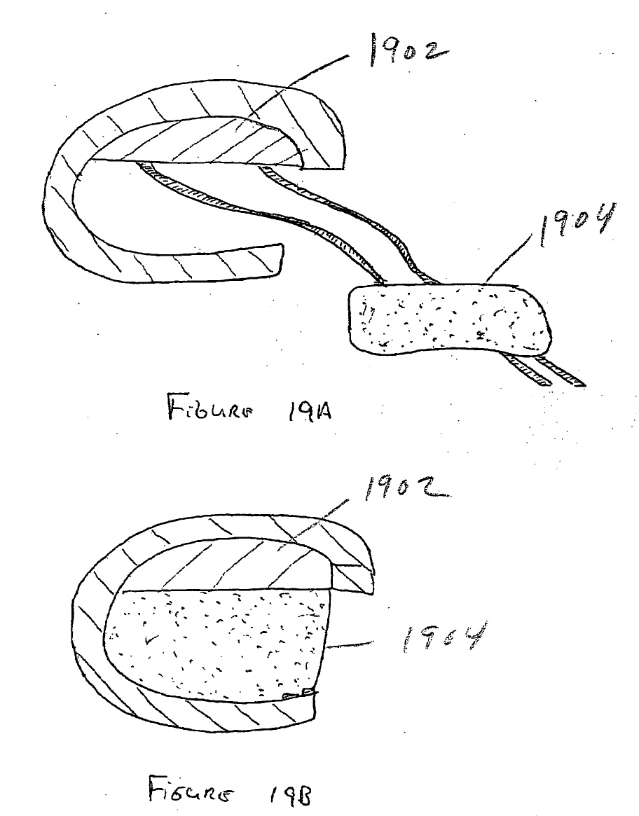

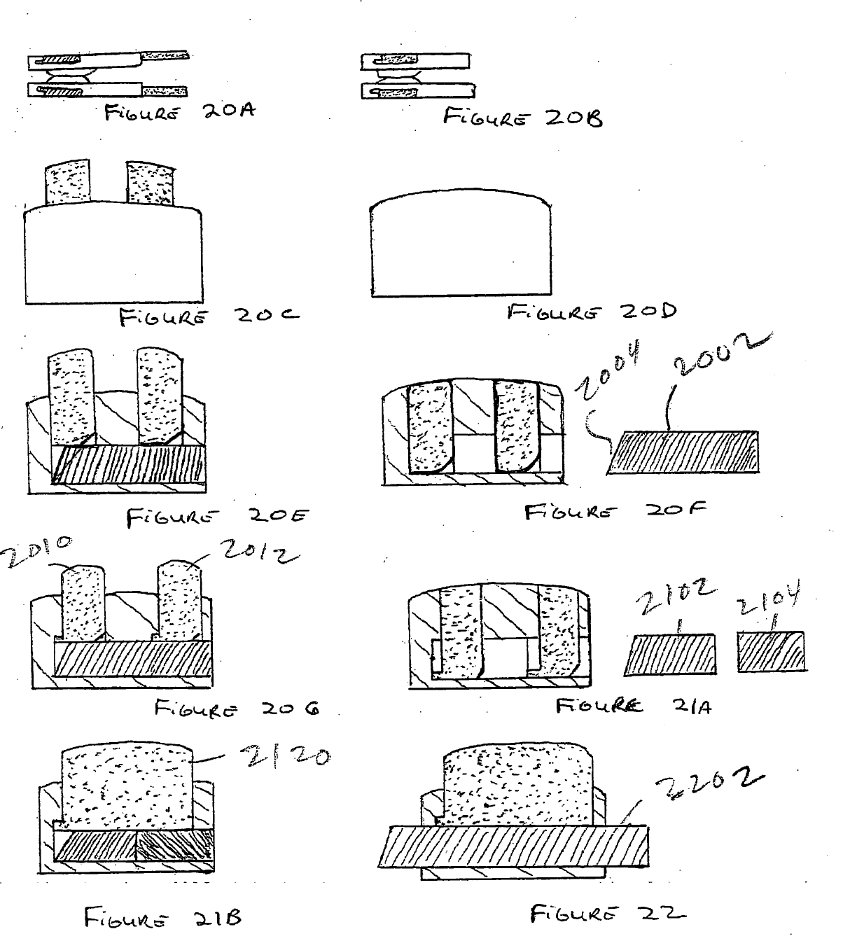

FIG. 5A is a view of the top of an alternative embodiment wherein the posterior component 500 contains a spherical joint component 502. FIG. 5B is a top, exploded view showing how a C-shaped component 504 passes through an opening in the second component. The C-shaped component has spring-like projections that snap into the second component. An optional latch may also be used to hold the components together. The C-shaped component is added to the second component after the second component has been inserted into the disc space. FIG. 5C is a lateral view of the assembled device drawn in FIG. 5A. The slots in the ADR endplate components are preferably angled to permit the anterior portions of the C-shaped components to contact the VEPs when the C-shaped components are fully inserted.

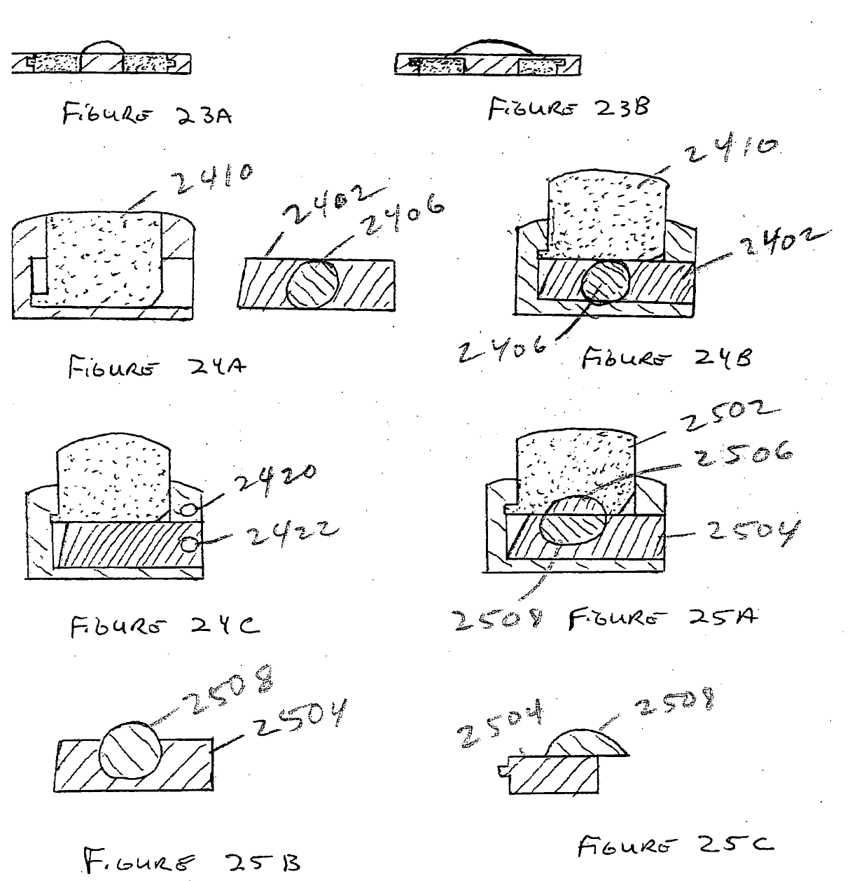

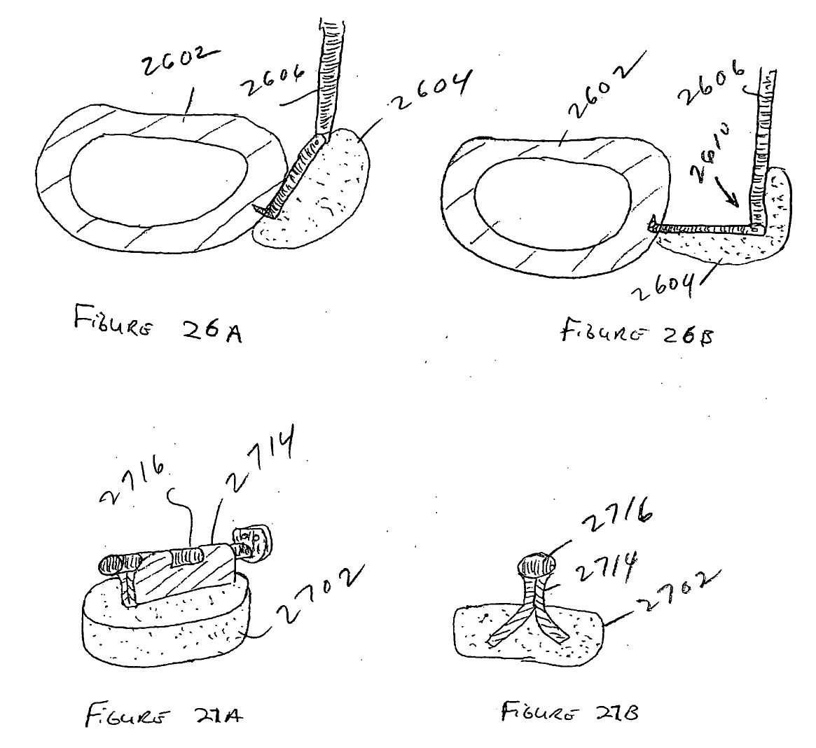

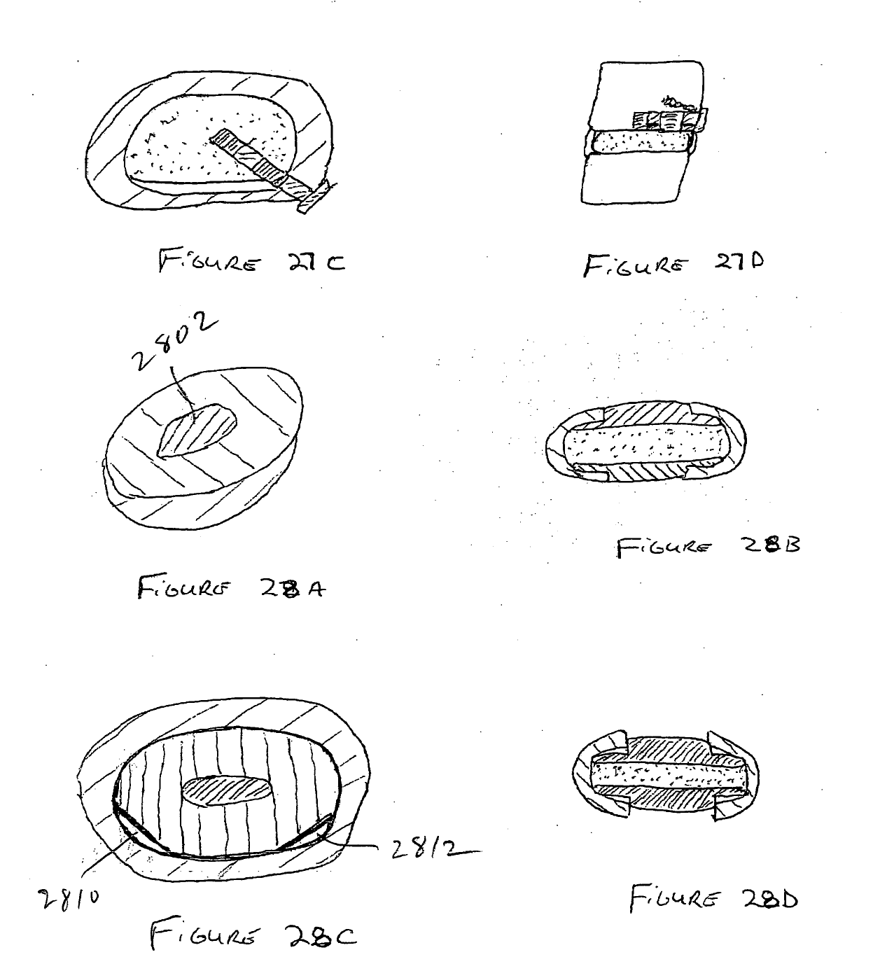

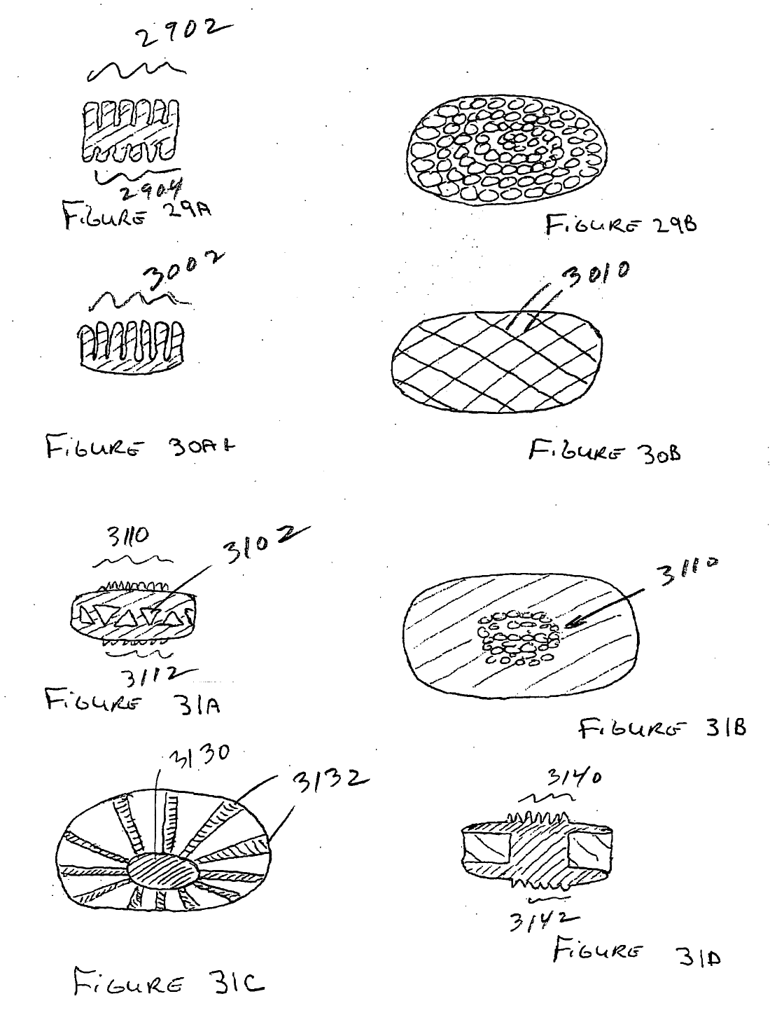

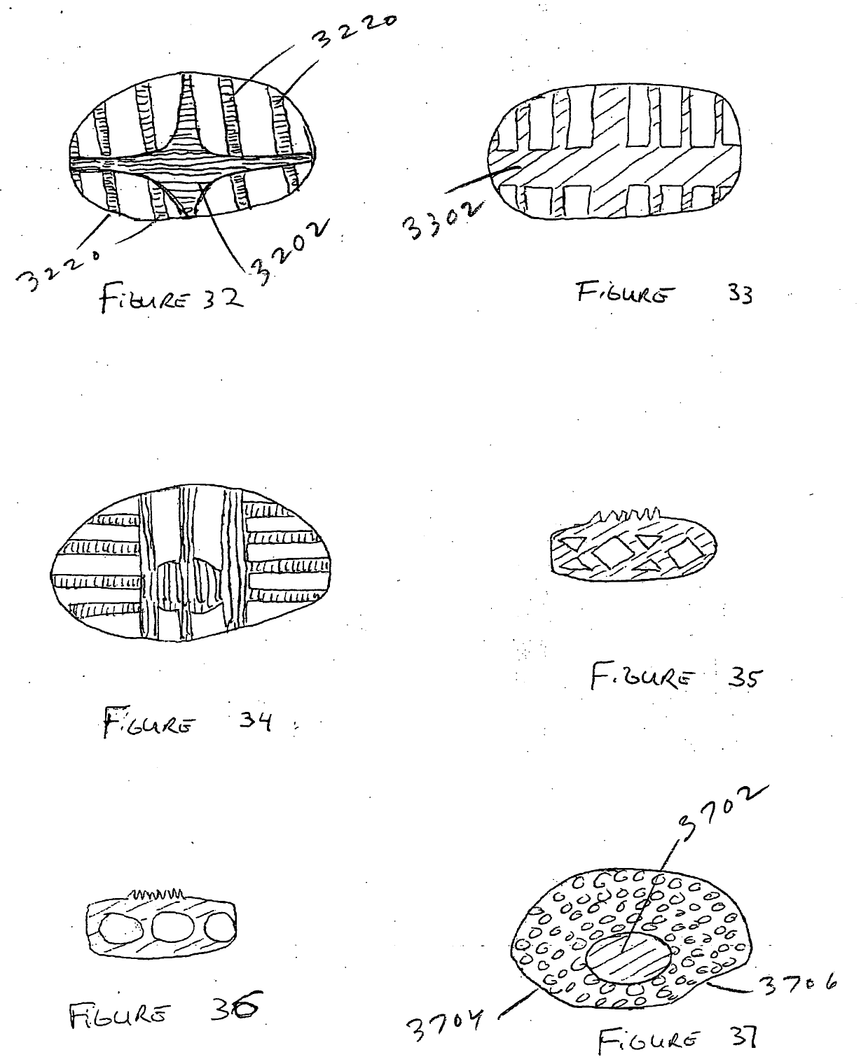

FIG. 6A is a top view of an alternative embodiment of the invention wherein the posterior half of the device has a spherical articulating component 602. The anterior and posterior halves of the device are connected with a hinge joint 604. FIG. 6B is an exploded view of the embodiment of the invention drawn in FIG. 6A. FIG. 6C is a view of the inferior surface of the unassembled device drawn in FIG. 6A. FIG. 6D is a view of the inferior surface of an assembled device drawn in FIG. 6A. A wedge component 610 expands the device in an anterior to posterior direction. A latch component can be used to hold the assembled device together. FIG. 6E is a lateral view of the embodiment of the invention drawn in FIG. 6A.

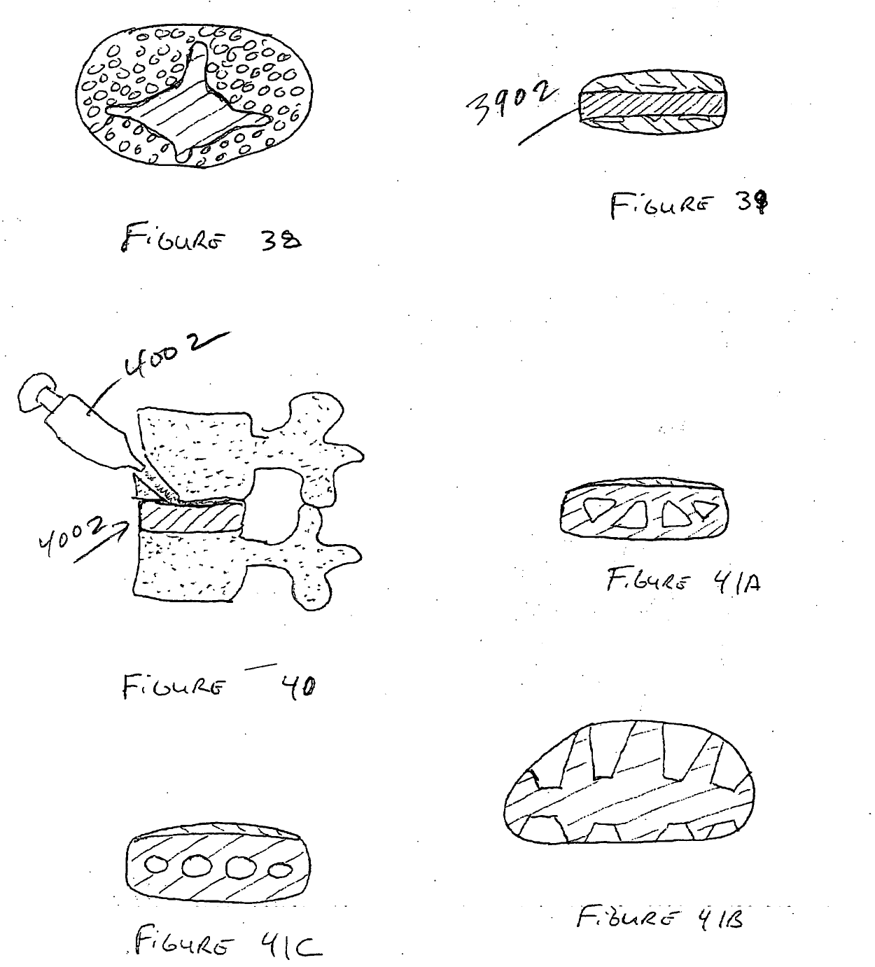

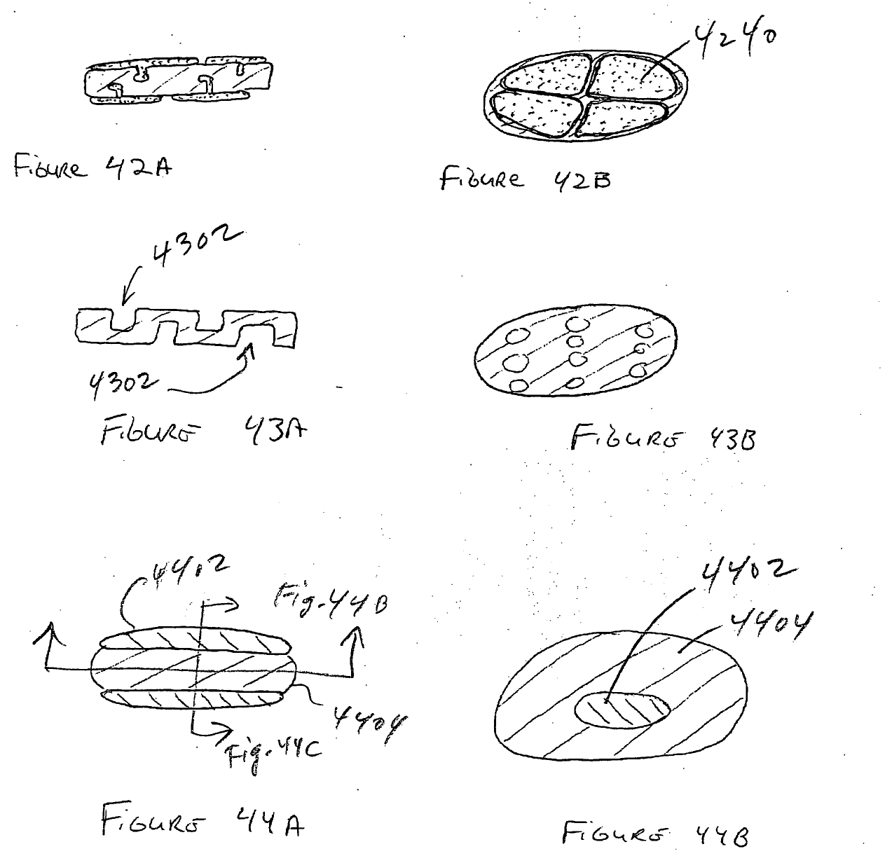

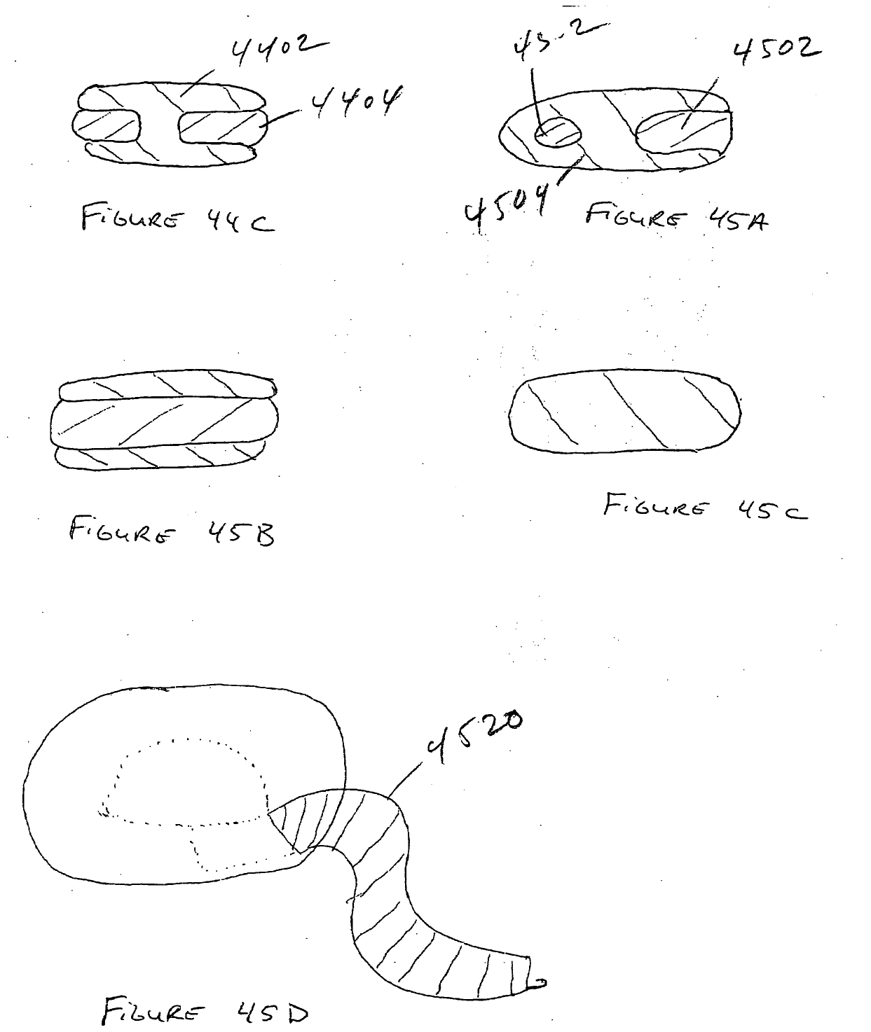

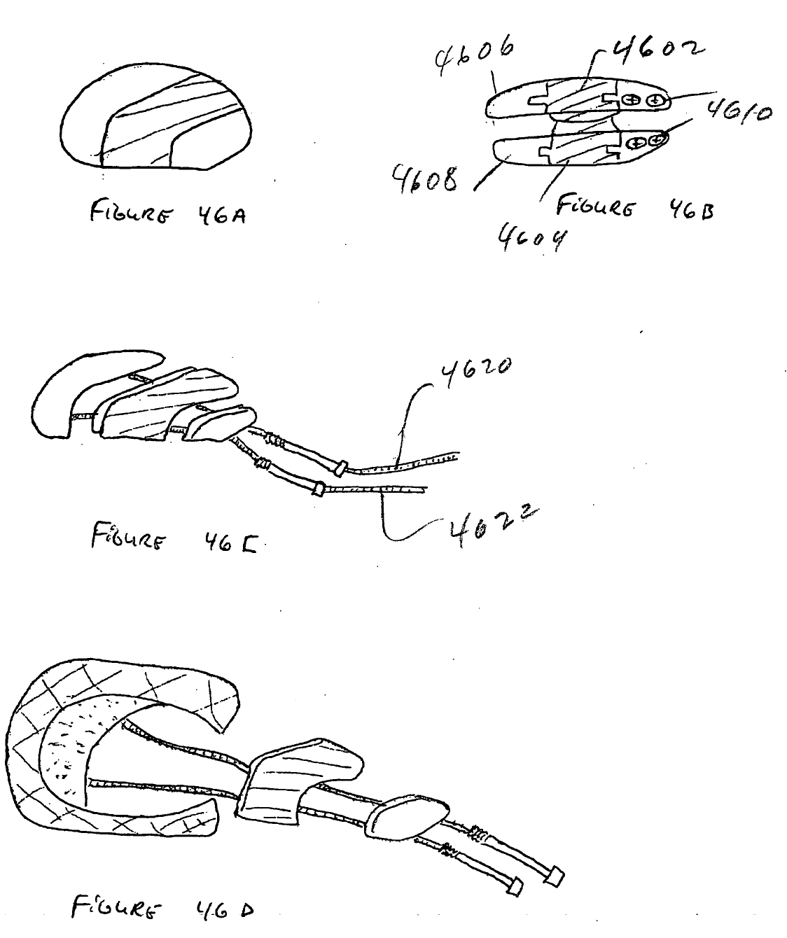

FIG. 7A is a top view of an alternative embodiment of the invention, and FIG. 7B is a view of the top of an alternative wedge component 702. The wedge component has an articulating surface 704. The wedge component may be used to expand the component drawn in FIG. 7A. FIG. 7C is a view of the top of the top of an embodiment of the device assembled by inserting the component drawn in FIG. 7B into the component drawn in FIG. 7A. The component drawn in FIG. 7B is inserted into the component drawn in FIG. 7A, after the 7A component is inserted into the disc space. FIG. 7D is a lateral view of the embodiment of the invention drawn in FIG. 7C.

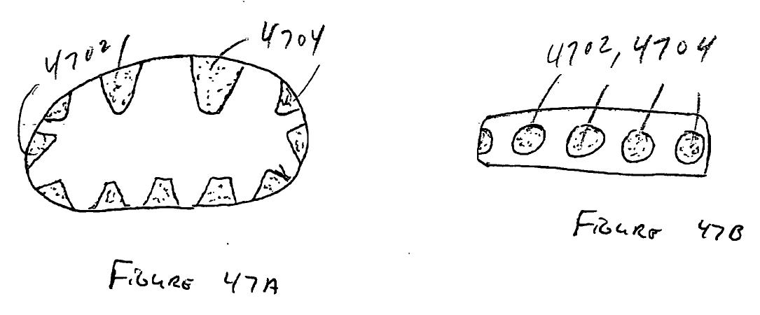

FIG. 8 is the view of the top of an alternative embodiment of the invention and an axial cross section of a disc. The drawing illustrates a TDR component 802 that is shaped to facilitate insertion into the disc space through a small opening in the AF. The component is rotated as it is inserted into the disc. The TDR has spherical or other shaped articulating surface(s).

FIG. 9 is a view of the top of an alternative embodiment of the invention and an axial cross section of a disc. Like device drawn in FIG. 8, the device 902 is shaped to facilitate insertion through a small opening in the AF. The device also has an articulating surface 904.

FIG. 10A is an exploded view of the top of an alternative embodiment of the invention including anterior and posterior components 1002, 1004 that slide relative to one another. A latch 1006 and screw 1008 can be used to hold the components in a fixed position. The posterior component has a spherical articulating surface 1010. Figure 10B is a view of the top of the embodiment of the invention drawn in FIG. 10A in its final shape. FIG. 10C is a view of the top of the embodiment of the invention drawn in FIG. 10A and an axial cross section of a disc. The drawing illustrates insertion of the TDR in a first shape that is different from the final shape. The first shape facilitates insertion of the TDR. FIG. 10D is a lateral view of the embodiment of the invention drawn in FIG. 10B.

FIG. 11A is an exploded view of the top of an alternative embodiment of the invention which includes an optional member 1102 that can be used to lock the anterior and posterior components 1104, 1106 together. FIG. 11B is a view of the top of the embodiment of the invention drawn in FIG. 11A. The outline of the locking member is represented by the dotted lines.

FIG. 12A is a view of the top of an alternative embodiment of the invention drawn in FIG. 11B. The anterior and posterior components 1202, 1204 articulate along a circular slot between the two components. FIG. 12B is a view of the top of the embodiment of the invention drawn in FIG. 12A. The two components are drawn in different positions than the positions drawn in FIG. 12A.

FIG. 13A is a view of the top of an alternative embodiment of the invention and an axial cross section of a disc. Two components 1302, 1304 project from the anterior portion of the device 1310. The device has been drawn in with the components in their extended position. FIG. 13B is an exploded view of the top of the embodiment of the invention drawn in FIG. 13A. The anterior components are retracted into the body of the posterior component. A wedge component 1320 is drawn to the right of the articulating component.

FIG. 13C is a view of the bottom of the embodiment of the invention drawn in FIG. 13B. FIG. 13D is a view of the bottom of the embodiment of the invention drawn in FIG. 13A. The wedge component forces the anterior components towards the front of the disc. The wedge component and the articulating component have a mechanism that fastens the components together.

FIG. 13E is an exploded view of the bottom of an alternative embodiment of the invention drawn in FIG. 13C. A single anterior component is seen retracted into the body of articulating component. FIG. 13F is view of the bottom of the embodiment of the invention drawn in FIG. 13E. The wedge component 1330 has been inserted to expand the TDR.

FIG. 14A is a view of the top of an alternative embodiment of the invention wherein two components 1402, 1404 are connected along a joint 1420 that extends diagonally across the device. FIG. 14B is a view of the top of the embodiment of the invention drawn in FIG. 14A. The components are drawn in a different position than the position of the components drawn in FIG. 14A. FIG. 14C is a view of the top of the embodiment of the invention drawn in FIG. 14A and an axial cross section of the disc. The TDR is drawn in a shape that facilitates insertion of the device into the disc.

FIG. 15A is a view of the top of an alternative embodiment of the invention drawn in FIG. 14A. The device is a different shape than the device drawn in FIG. 14A when the articulating components 1502, 1504 are aligned. FIG. 15B is a view of the top of an alternative embodiment wherein the articulating surface 1510 is limited to the posterior component. Although in all embodiments spherical articular surfaces are preferred, other surfaces with non-spherical and/or compound surfaces may alternatively be used.

FIG. 16A is an exploded view of the top of an alternative embodiment of the invention including a projection 1602 from one component 1604 fits into a slot in the second component 1610. FIG. 16B is an exploded view of the top of the embodiment of the invention drawn in FIG. 16A and an axial cross section of a disc. The first component has been inserted in the disc space.

FIG. 16C is a view of the top of the embodiment of the invention drawn in FIG. 16B and an axial cross section of a disc. The TDR has been drawn in its final shape. The articulating surface is shown at 1620. FIG. 16D is a view of the top of an alternative embodiment of the anterior TDR component drawn in FIG. 16A. The component has features 1620 that fasten the TDR components together.

FIG. 17A is an exploded view of the top of an alternative embodiment of the invention drawn in FIG. 16A. Both the anterior and the posterior components 1702, 1704 are figured to fasten together using a cable 1710 that passes from one component through the second component. The cables can be used to pull the components together. The cables facilitate fastening the components together while the components are within the disc space. FIG. 17B is a top view showing the components fastened together. The cables may optionally crimped to help hold the components together.

FIG. 18A is an exploded view of an alternative embodiment of the invention wherein cables 1806 are used to pull two or more components 1802, 1804 together. FIG. 18B is a view of the top of the embodiment of the invention drawn in FIG. 18A. The components are drawn in their assembled position.

FIG. 19A is an exploded view of an alternative embodiment of the invention and an axial cross section of the disc. The first component 1902 has been inserted into the disc. FIG. 19B is a view of the top of the embodiment of the invention drawn in FIG. 19A and an axial cross section of the disc. The components 1902, 1904 are drawn in their assembled position. Component 1904 may be an articulating component.

FIG. 20A is lateral view of an alternative embodiment of the invention with the TDR drawn in its extended position. FIG. 20B is a lateral view of the embodiment of the invention drawn in FIG. 20A with the TDR drawn in its contracted position. FIG. 20C is a view of the top of the embodiment of the invention drawn in FIG. 20A with the TDR drawn in its extended position. FIG. 20D is a view of the top of the embodiment of the invention drawn in FIG. 20B with the TDR drawn in its contracted position.

FIG. 20E is a view of the bottom of the embodiment of the invention drawn in FIG. 20C. The TDR was drawn in its extended position. FIG. 20F is an exploded view of the bottom of the embodiment of the invention drawn in FIG. 20D. The TDR is drawn in its contracted position. The wedge component is inserted into the TDR to force it into its extended position. The wedge component 2002 is inserted into the TDR after the TDR is placed into the disc space. The leading edge 2004 of the wedge component is beveled to push the anterior components towards the front of the TDR. The posterior corners of the anterior components are beveled to cooperate with the wedge component. The wedge component may be reversibly fastened to the TDR.

FIG. 20G is a view of the bottom of an alternative embodiment of the invention including anterior components with side projections 2010, 2012. The projections cooperate with the TDR endplates to limit how far the anterior components project from the anterior portion of the TDR.

FIG. 21A is an exploded view of bottom of an alternative embodiment of the invention with two wedge components 2102, 2104 used to advance retractable anterior components. Two wedge components require less muscle retraction to insert them into the TDR than a single longer component 2010 requires to insert into the TDR. FIG. 21B is a view of the bottom of an alternative embodiment of the invention drawn in FIG. 21A. A single retractable component 2120 projects anterior to the TDR.

FIG. 22 is a view of the bottom of an alternative embodiment of the invention wherein the wedge component 2202 is wider than the TDR. The wedge component increases the area of contact with the vertebral endplates (VEPs).

FIG. 23A is an anterior view of an embodiment of the invention wherein the retractable members extend from the top to the bottom of each TDR EP. FIG. 23B is an anterior view of an alternative embodiment of the invention wherein the retractable members do not extend all the way to the top of the EP. The retractable members also extend below or above a portion of the concave and convex articulating surfaces.

FIG. 24A is an exploded view of top of an alternative embodiment of the invention having wedge components 2402 that contain the an articulating surface 2406 used to form a joint between the TDR EPs. FIG. 24B is a view of the top of the embodiment of the invention drawn in FIG. 24A. The retractable component 2410 has been drawn in its extended position. FIG. 24C is view of the bottom of an alternative embodiment of the invention wherein the retractable and articulating components have holes 2420, 2422. Screws can be placed through the holes to fasten the TDR to the vertebrae. The screws also hold the TDR components together.

FIG. 25A is a view of the top of an alternative embodiment of the invention drawn wherein the retractable component 2502 and the wedge component 2504 contain portions of the articulating surfaces 2506, 2508. FIG. 25B is a view of the top of the wedge component drawn in FIG. 25A. FIG. 25C is a lateral view of the wedge component drawn in FIG. 25B.

FIG. 26A is an axial cross section of a disc 2602, a psoas muscle 2604, and a novel articulating retractor 2606. The end of the retractor is placed between the psoas muscle and the disc. FIG. 26B is an axial cross section of a disc, a psoas muscle, and the embodiment of the invention drawn in FIG. 26A. The retractor has been adjusted to increase the space between the psoas muscle and the side of the disc. The retractor may contain a hinge joint 2610 between different components of the retractor.

FIG. 27A is an oblique view of a nucleus replacement (NR) according to the invention having a cushion component 2702, a tether component 2714, and rod component 2716. The cushion component fits within the disc space. The tether component passes through slot cut into a vertebra above or below the NR. The rod component passes into a hole drilled into the vertebra above or below the NR.

FIG. 27B is a coronal cross section the embodiment of the invention drawn in FIG. 27A. The tether component 2714 is embedded into the cushion component. The tether component also passes around the rod. The tether component is preferably made of a relatively inelastic material such as nylon, Dacron, Gortex, or other woven fabric. The cushion component is preferably made of an elastomer such as Elasthane, Pellothane, C-Flex, Biomer, etc. The rod component is preferably made of titanium. The surface of the rod is preferably treated to facilitate bone in-growth.

FIG. 27C is an axial cross section of the disc and a view of the top of the embodiment drawn in FIG. 27A. The NR is positioned anterior to the posterior portion of the AF. The tether prevents the NR from moving against the posterior portion of the AF. FIG. 27D is a coronal cross section of the spine and an anterior view of the embodiment of the invention drawn in FIG. 27C.

FIG. 28A is an oblique view of an alternative embodiment of the invention, and FIG. 28B is a coronal cross section of the embodiment of the invention drawn in FIG. 28A. The core 2802 of the NR is preferably made of polymer with a lower durometer than the durometer of the material used for the shell 2804. The center of the top and the bottom of the shell is separated from the remainder of the shell. The core is preferably attached to the “caps” of shell on the top and bottom of the core. In the preferred embodiment the core and the caps area not attached to the shell. The device is configured to allow the shell to expand without stretching the “caps”.

FIG. 28C is an axial cross section of a disc and a view of the top of the embodiment of the invention drawn in FIG. 28A. The posterior corners 2810, 2812 of the NR are beveled to prevent the NR from applying pressure on the posterior-lateral portions of the AF. FIG. 28D is a coronal cross section of the embodiment of the invention drawn in FIG. 28B. Loads have been applied to the caps of the device. The figure illustrates movement between the caps and the shell of the device.

FIG. 29A is a lateral view of an alternative embodiment of the invention with cylinder-shaped projections on the top 2902 and the bottom 2904 of the device. Therapeutic material such as collagen, hydrogel, allograft tissue, dehydrated tissue, bone growth material, glycoproteins including chondroitin sulphate and keratan sulphate or other material may be placed over the NR and between the projections in the NR. The therapeutic material could contain cytokines such as TGF-B, PDGF, VEGF, BMP, MSCF, IGF, etc could be released from the therapeutic material. The therapeutic material and/or cytokines could facilitate healing of the disc, including tears in the AF. The therapeutic material could also improve the fit between the NR and the VEP. The therapeutic material could cause the VEPs to remodel or grow to fit the NR. The therapeutic material could also cause fluid movement into and out of the disc space. For example, dehydrated collagen could imbibe fluids. The fluid could be forced into and out of the collagen as the NR is loaded and unloaded. FIG. 29B is a view of the top of the embodiment of the invention drawn in FIG. 29A.

FIG. 30A is a lateral view of an alternative embodiment of the invention wherein projections 3002 are limited to one side of the NR. The stiffness of the NR could be varied by changing the diameter of the projections, the length of the projections, the space between the projections, the thickness of the disc-like component below and/or above the projections, the durometer of the material, and the type of material. FIG. 30B is a view of the top of the embodiment of the invention drawn in FIG. 30A. Multiple incisions 3010 are made on the top of the device to create diamond-shaped projections.

FIG. 31A is lateral view of an alternative embodiment of the invention in the form of a device with holes that extend into the sides of the NR. The holes 3102 are preferably triangular in cross section, and the top and the bottom of the NR have small projections 3110, 3112. FIG. 31B is a view of the top of the embodiment of the invention drawn in FIG. 31A. Projections 3110 may be seen on the top of the NR.

FIG. 31C is an axial cross section of an embodiment of the invention wherein holes pass from the periphery of the device to a solid core within the device. The solid core 3130 preferably located in the posterior portion of the NR. The cross sections of the walls of the holes are represented by the radial spokes 3132. FIG. 31D is a coronal cross section of the embodiment of the device drawn in FIG. 31C. The cross section was taken through the solid core of the NR. The projections 3140, 3142 from the top and the bottom of the device can deform to fit irregularities in the V. EPs.

FIG. 32 is an axial cross section through an alternative embodiment of the invention. The solid core of the device is represented by the four-pointed star-like portion 3202. The points of the “star” taper as they course to the edges of the NR. The tapered portions of the core facilitate flexion, extension, and lateral bending of the spine. The walls of the holes are represented by the areas 3220. The holes within the device and the space above, below, and around the NR may be filled with therapeutic material as described in the text of FIG. 29A. FIG. 33 is an axial cross section through an alternative embodiment wherein the core of the device is represented by the thicker, shaped component 3302 in the interior of the NR.

FIG. 34 is an axial cross section of an alternative embodiment of the invention wherein the holes in the NR course form left to right and from anterior to posterior. FIG. 35 is a lateral view of the embodiment of the invention drawn in FIG. 34. FIG. 36 is a lateral view of an alternative embodiment of the invention wherein the holes are circular in cross section.

FIG. 37 is a view of the top of an alternative embodiment of the invention including a large projection 3702 from the top and bottom of the posterior portion of the NR. The posterior-lateral corners 3704, 3706 of the device are beveled to prevent pressure on the posterior-lateral portions of the AF.

FIG. 38 is a view of the top of an alternative embodiment of the invention wherein large four-pointed, star-like components project from the top and/or the bottom of the NR. FIG. 39 is lateral view of an alternative embodiment of the invention wherein an elastic band 3902 surrounds the periphery of the NR. The band helps hold therapeutic material in the holes in the device. The band may be porous to facilitate fluid movement into and out of the therapeutic material.

FIG. 40 is a sagittal cross section of the spine, a NR or TDR 4002, and alternative embodiment of the invention. A syringe 4002 is used to inject therapeutic material into the disc space above the ADR. The therapeutic material is injected through a hole in the vertebra. The therapeutic material fills spaces between the NR and the VEP. The therapeutic material may include in-situ curing polymers such as polyurethane.

FIG. 41A is an anterior view of an alternative embodiment of the invention, and FIG. 41B is an axial cross section through the embodiment of the invention drawn in FIG. 41A. The holes 4102 in the NR are tapered such that the anterior portions of the anterior holes are wider than the posterior portions of the anterior holes. Similarly, the posterior portions of the posterior holes are wider than the anterior portions of the posterior holes. The NR is preferably thickest in the posterior portion of the device. The holes in the NR increase the flexibility of the device. The design enables the use of materials that are more durable and less flexible. The design facilitates spinal flexion, extension, and lateral bending. As described in the text of FIG. 29A, the hole in the device, as well as the disc space around the device may be filled with therapeutic material(s).

FIG. 41C is an anterior view of an alternative embodiment of the invention drawn in FIG. 41A. The NR has cone-shaped holes in the anterior and the posterior portions of the device. The NR may also have cone shaped holes on the sides of the device. The axial cross section of the device is the same as that drawn in FIG. 41B.

FIG. 42A is a lateral view of an alternative embodiment of the invention drawn in FIG. 41A. The top and the bottom of the device are covered or partially covered with hard plates. The plates could be made of metal, ceramic, or other material that has better wear characteristics than the cushion component. The plates may be snapped into the cushion component with plastic deformation of the cushion component. FIG. 42B is a view of the top the embodiment of the invention drawn in FIG. 42A. Alternative configurations of device may include one or more plates such as 4240 on the top or the bottom of the NR.

FIG. 43A is a lateral view of an alternative embodiment of the invention wherein the NR has holes or slots 4302 on the top and the bottom of the device. BMP-soaked sponges (or other beneficial substances) may be placed into the holes of the device. Bone could grow from the vertebrae and into the holes of the NR. The bone projections could help stabilize the device in the disc space. FIG. 43B is a view of the top of the embodiment of the invention drawn in FIG. 43A.

FIG. 44A is a view of the front of an alternative embodiment of the invention wherein, like the NR in FIG. 41A, the NR is stiffer in its interior than around the periphery of the NR. The central portion and the top and bottom of the NR are made of a stiffer material than the material that surrounds the periphery of the device.

FIG. 44B is an axial cross section of the embodiment of the invention drawn in FIG. 44A. The core 4402 is made of a material with higher durometer than the material used to form a ring 4404 around the core. FIG. 44C is a sagittal cross section of the embodiment of the invention drawn in FIG. 44B. The material that forms the top, bottom, and pedestal of the NR is stiffer than the material used to form the ring around the periphery of the device.

FIG. 45A is a lateral view of an alternative embodiment of the invention wherein the softer material 4502 passes through a tube-shaped opening in the posterior portion of the device 4504. The stiff material in the tube prevents the softer, more flexible material from applying pressure to the posterior AF. FIG. 45B is a view of the front of the embodiment of the invention drawn in FIG. 45A.

FIG. 45C is a view of the back of the embodiment of the invention drawn in FIG. 45A. FIG. 45D is a view of the top of the embodiment of the invention drawn in FIG. 45A. The softer material 4520 may be added to the harder, less flexible, component after the stiffer component is positioned in the disc space. The edges of the stiffer component may be folded to facilitate insertion of the device. The softer material around the core of the device may cure in-situ. Several pieces of the softer material may be inserted after insertion of the harder core of the device. For example, beads of softer material may be added through a slit in a tube that courses around the periphery of the device. Hydrogel may be used as the softer material that surrounds the periphery of the NR. The hydrogel could imbibe fluid after placement of the device. The expansion of the hydrogel could be limited by tube that surrounds the hydrogel. The opening in the tube could be sealed in-situ with heat, ultrasound, or a laser.

FIG. 46A is a view of the top of an alternative embodiment having upper and the lower ADR endplates (EPs) that are assembled from three components. FIG. 46B is an anterior view of the embodiment of the invention drawn in FIG. 46A. The central components 4602, 4604 of the upper and the lower ADR EPs 4606, 4608 articulate with one another. The components preferably articulate through a spherical joint. The three components of the upper ADR EP and the three components of the lower ADR EP are connected with tongue and groove joints and screws 4610.

FIG. 46C is an exploded view of the top of the embodiment of the invention drawn in FIG. 46A. Flexible cords 4620, 4622 pass from the first ADR EP component and through the second and third ADR EP components. The flexible cords are used to guide the tongue of one component into the groove or slot of a second ADR EP component. The flexible cords also guide cannulated screws into the ADR EP components. The invention facilitates assembly of the ADR within the AF of the disc.

FIG. 46D is an axial cross section of a disc and an exploded view of the top of the embodiment of the invention drawn in FIG. 46C. The first component of the upper ADR EP and the first component of the lower ADR EP (hidden in the drawing by the component from the upper ADR EP) have been inserted into the disc. The two components are preferably held relative to one another by a resorbable component. For example the two components may be held near each other by ice which melts after insertion, thus allowing movement between the components.

FIG. 47A is an axial cross section of an alternative embodiment of the invention wherein lateral portions of the ADR have additional conical shaped holes. The additional holes decrease the stiffness of lateral portions of the device. The holes are represented by areas 4702, 4704, etc. FIG. 47B is a view of the anterior-lateral portion of the invention drawn in FIG. 47A.

I claim:

1. An intradiscal device capable of posterior-lateral insertion, comprising:
an anterior component;
a posterior component; and
an assembly component that either expands the components in an anterior or posterior direction or connects the components in situ.

2. The intradiscal device of claim 1, wherein the posterior component has a raised articulation surface.

3. The intradiscal device of claim 1, wherein the posterior component has a raised spherical articulation surface.

4. The intradiscal device of claim 1, wherein the assembly component is pushed into the posterior component causing the anterior component to slide out of the posterior component in the anterior direction.

5. The intradiscal device of claim 1, wherein the assembly component is a cable used to bring the anterior and posterior components together within an intradiscal space.

6. The intradiscal device of claim 1, wherein the anterior and posterior components each provide a portion of a raised articulating surface.

7. The intradiscal device of claim 1, wherein all of the components are inserted in situ between previously placed upper and lower endplate components.

8. An improved implant, comprising:
a device configured for insertion into a joint or intradiscal space leaving a void; and
therapeutic or other beneficial material within the void.

9. The improved implant of claim 8, wherein the therapeutic or other beneficial material includes one or more of the following:
collagen, hydrogel, allograft tissue, dehydrated tissue, bone growth material, glycoproteins including chondroitin sulphate and keratan sulphate.

10. The improved implant of claim 8, wherein the therapeutic or other beneficial material cytokines such as TGF-β, PDGF, VEGF, BMP, MSCF, or IGF.
